(19) United States
(12) Patent Application Publication
(10) Pub. No.: US 2026/0294990 A1
(43) Pub. Date: Oct. 1, 2026

(54) Multi Ingredient Natural Products Composition And Method Of Use

(71) Applicant: Visionary Ventures LLC, Spearfish, SD (US)

(72) Inventors: Mark R. Gordon, Spearfish, SD (US); Veronica Davis-Gordon, Vista, CA (US)

(73) Assignee: Visionary Ventures LLC, Vista, CA (US)

(21) Appl. No.: 19/096,248

(22) Filed: Mar. 31, 2025

(51) Int. Cl. A61K 36/23; A61K 31/05; A61K 31/19; A61K 31/352; A61K 31/7048; A61K 36/53; A61K 36/82; A61K 36/9068

(52) U.S. Cl. CPC A61K 36/23; A61K 31/05; A61K 31/19; A61K 31/352; A61K 31/7048; A61K 36/53; A61K 36/82; A61K 36/9068

(57) ABSTRACT

A multi-ingredient natural product composition for enhancing longevity and cellular health includes a formulation comprising a mixture of natural products comprising at least five natural products selected from the group including gotu kola extract, green tea extract, ginger root extract, rosemary extract, resveratrol, ursolic acid, isoquercetin, and luteolin. Also included are one or more of a liquid, an excipient, and a device configured for rendering the mixture of natural products suitable for therapeutic delivery to a subject. The formulation is configured for improving a biological process in the subject.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to natural products compositions and more particularly pertains to a new natural product composition for enhancing longevity and cellular health. While a variety of natural products compositions are known in the prior art, there remains a need for novel natural products compositions that are specifically directed to the enhancement of longevity and cellular health.

### (2) Description of Related Art Including Information Disclosed Under 37 CFR 1.97 and 1.98

The prior art relates to natural products compositions, but which do not include a mixture of natural products comprising at least five natural products selected from the group consisting of gotu kola extract, green tea extract, ginger root extract, rosemary extract, resveratrol, ursolic acid, isoquercetin, and luteolin. Conventional approaches to longevity and cellular health frequently rely on singular mechanisms, resulting in incomplete or narrowly focused benefits that fail to address the complex network of aging-associated pathways such as oxidative stress, inflammatory cascades, and metabolic dysregulation. These approaches do not adequately target a broad spectrum of pathways involved in cellular resilience and repair.

Many existing interventions do not adequately modulate critical signaling pathways, such as Nrf2, AMPK, sirtuins, and mTOR, to produce a robust and synergistic effect, leading to suboptimal outcomes when attempting to delay age-related decline or enhance cellular resilience and repair mechanisms. Furthermore, other key signaling pathways, including telomere maintenance, nitric oxide synthase (NOS), NF-κB, MAPK, and PI3K/AKT, are often neglected despite their central roles in cellular health, inflammation, and metabolic regulation.

Products currently available on the market often overlook the necessity of combinatorial and synergistic action on vascular function, cognitive performance, energy metabolism, and cellular renewal, thereby limiting their capacity to comprehensively support systemic health, mitigate chronic inflammation, and foster overall well-being throughout the aging process. For example, the inhibition of pro-inflammatory cytokines, including TNF-α, interleukin-1β (IL-1β), interleukin-6 (IL-6), interleukin-11 (IL-11), and interleukin-17 (IL-17), is rarely addressed in existing products, despite the profound impact of chronic inflammation on aging.

Additionally, mechanisms such as collagen synthesis and angiogenesis, facilitated by compounds like gotu kola, are critical yet underutilized strategies for enhancing cellular and tissue repair processes. Similarly, targeting pathways like cyclooxygenase-2 (COX-2) inhibition and lipopolysaccharide (LPS) suppression provides significant opportunities for reducing oxidative damage and inflammatory cascades.

### Brief Summary of the Invention

An embodiment of the disclosure meets the needs presented above by generally comprising a formulation, which in turn comprises a mixture of natural products comprising at least five natural products selected from the group comprising gotu kola extract, green tea extract, ginger root extract, rosemary extract, resveratrol, ursolic acid, isoquercetin, and luteolin. The formulation also includes one or more of a liquid, an excipient, and a device configured to render the formulation suitable for therapeutic delivery to a subject. The formulation thus is configured to improve a biological process in the subject leading to enhanced longevity and cellular health for the subject. Each natural product is present in specific concentrations to synergistically activate pathways such as Nrf2, AMPK, sirtuins, and mTOR inhibition. The formulation reduces oxidative stress, inflammation, and cellular damage while enhancing vascular function, cognitive health, energy metabolism, and cellular renewal. The formulation supports hair growth, enhances clear skin and complexion, and creates hormone balance in men and women. The formulation provides a comprehensive solution for improving longevity, mitigating age-related decline, and promoting overall physiological health.

Another embodiment of the disclosure includes a method of treating a subject with a mixture of natural products to improve a biological process in the subject. The method entails provision of the mixture of natural products, as per the disclosure above, and the one or more of the liquid, the excipient, and the device. Upon producing a formulation, it can be delivered to the subject to improve the biological process.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a block diagram of a multi-ingredient natural products composition according to an embodiment of the disclosure.

FIG. 2 is a flowchart for a method utilizing an embodiment of the disclosure.

### DETAILED DESCRIPTION OF THE INVENTION

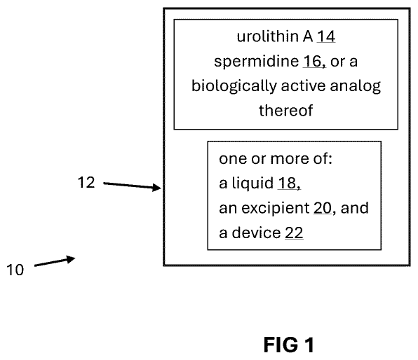

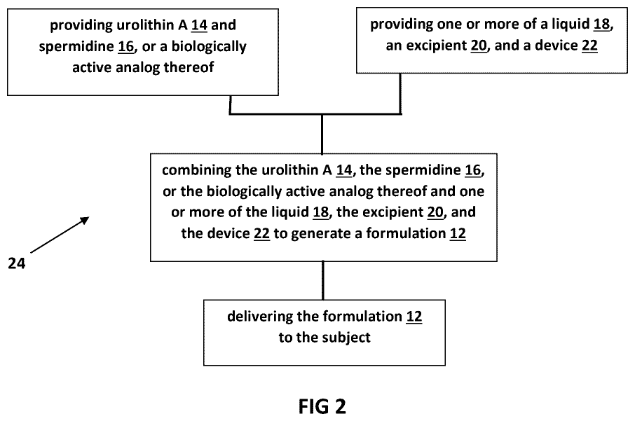

With reference now to the drawings, and in particular to FIGS. 1 and 2 thereof, a new natural products composition embodying the principles and concepts of an embodiment of the disclosure and generally designated by the reference numeral 10 will be described.

As best illustrated in FIGS. 1 and 2, the multi-ingredient natural products composition 10 generally comprises a formulation 12, which in turn comprises a mixture of natural products 14 comprising at least five natural products selected from the group consisting of gotu kola extract 16, green tea extract 18, ginger root extract 20, rosemary extract 22, resveratrol 24, ursolic acid 26, isoquercetin 28, and luteolin 30. The formulation 12 also includes one or more of a liquid 32, an excipient 34, and a device 36 configured to render the formulation 12 suitable for therapeutic delivery to a subject. The formulation 12 thus is configured to improve a biological process in the subject leading to enhanced longevity and cellular health for the subject.

The one or more of the liquid 32, the excipient 34, and the device 36 of the multi-ingredient natural products composition 10 may be any liquid 32, excipient 34, or device 36 that facilitates the transport, release, and subsequent administration of the mixture of natural products 14 to the subject, encompassing various routes such as oral, parenteral, transdermal, inhalational, or otherwise, wherein the design parameters, including dosage form, release profile, and composition of excipients 34 are selected to optimize bioavailability, stability, subject compliance, and therapeutic efficacy.

For example, the formulation 12 may be rendered as a pill, a tablet, a capsule, or other solid oral dosage form comprising one or more excipients 34 such as, but not limited to, binding agents, disintegrants, inert or minimally active fillers, or the like, which can be compressed, encapsulated, or the like, to produce a stable unit that is convenient for oral administration. Inert or minimally active fillers include, but are not limited to, lactose, cellulose derivatives, starch, calcium phosphate salts, various sugars, various sugar alcohols, and numerous other pharmaceutically acceptable inert or minimally active fillers that may be used depending on the dosage form. Inert or minimally active fillers are selected based on their ability to ensure content uniformity, to support tablet binding or flow properties, and to maintain chemical stability. By modulating compressibility, dissolution rates, or bioavailability, the inert or minimally active fillers help produce a consistently dosed and stable formulation 12 that adheres to stringent regulatory, safety, and efficacy requirements.

In some embodiments, the formulation 12 is rendered as a time-release capsule or transdermal patch to ensure sustained bioavailability of the mixture of natural products 14. Additionally, the formulation 12 may be rendered deliverable using nanoencapsulation or liposomal delivery systems to enhance the absorption of hydrophobic natural product(s) in the mixture of natural products 14, such as, but not limited to, ursolic acid 26 and isoquercetin 28. The formulation 12 ensures effective systemic distribution and targeted action in key tissues such as skeletal muscle, brain, and vascular endothelium.

In other embodiments, the formulation 12 may be rendered as a solution, suspension, or the like, which may require dissolving the mixture of natural products 14 in a liquid 32 that is a pharmaceutically acceptable solvent system or uniformly dispersing the mixture of natural products 14 in a liquid 32 that acts as a suspension medium, potentially with surfactants, viscosity-modifying polymers, or stabilizing excipients 34. The resulting formulation 12 can be administered orally, parenterally, or via other routes as deemed appropriate.

The excipient 34 may comprise a flavoring agent, which is incorporated into the formulation 12 to enhance flavor or to mask inherent flavor, thereby improving palatability and subject compliance. The flavoring agent may be derived from natural sources, such as essential oils and fruit extracts, or they may be synthesized chemically. The flavoring agent is selected based on their stability during manufacturing and throughout the product's shelf life. The flavoring agent also can include sweeteners, specialized taste-masking agents, and synthetic or natural flavor compounds, all of which are designed to impart or modify sensory attributes such as sweetness, bitterness, or acidity while remaining compatible with mixture of natural products 14 and other excipients 34.

The excipient 34 can also comprise a preservative, which may have antimicrobial or fungistatic properties. The preservative is incorporated into the formulation 12 to inhibit microbial growth and maintain integrity of the formulation 12 throughout its shelf life. Common examples of the preservative include, but are not limited to, parabens, benzoic acid, and sorbic acid, along with their corresponding salts. Additionally, phenolic compounds that effectively reduce or eliminate bacterial, fungal, or other microbial contamination can serve as the preservative. The preservative may be selected based on its spectrum of activity, stability within the formulation 12, and regulatory acceptance in accordance with established safety and efficacy guidelines. Formulators often optimize the concentration and compatibility of the preservative to ensure that it does not adversely affect the mixture of natural products 14 or the taste or the physicochemical properties of the formulation 12 while preserving overall quality and subject acceptability properties of the formulation 12.

The mixture of natural products 14 may include gotu kola extract 16, which typically is obtained from the aerial parts of centella asiatica, a plant traditionally employed in various regions for its purported therapeutic and longevity-promoting properties. The process of preparing gotu kola extract 16 commonly involves comminuting or pulverizing the dried plant matter, introducing an appropriate solvent, such as aqueous, alcoholic, or hydroalcoholic solutions, and subjecting the mixture to filtration, evaporation under reduced pressure, and other optional refinement steps, resulting in a final product enriched with triterpenoid saponins (e.g., asiaticosides, madecassosides) and other constituents. Gotu kola extract 16 may have many effects on human physiology, including the support of collagen synthesis, modulation of inflammatory pathways, and enhancement of microcirculatory parameters, which are hypothesized to benefit tissue repair and cellular vitality. Gotu kola extract 16 has also been shown to promote angiogenesis, the formation of new blood vessels, through its enhancement of prolyl hydroxylase activity. In one embodiment, the gotu kola extract 16 may be 40% asiaticosides by weight, a purity level that may be achieved with high-performance liquid 32 chromatography (HPLC).

The mixture of natural products 14 may include green tea extract 18, which typically is a concentrated preparation of bioactive compounds sourced from the leaves of Camellia sinensis, which is enriched in polyphenolic constituents such as catechins, epicatechins, and epigallocatechin gallate (EGCG). Green tea extract 18 is commonly manufactured via processes that involve harvesting and drying of the leaves followed by solvent-based extraction using water, ethanol, or other pharmaceutically acceptable solvents under controlled temperature and pH conditions to optimize both yield and purity. After extraction, the resulting solution is subjected to filtration or centrifugation to remove insoluble matter and subsequent concentration steps, such as vacuum evaporation or spray drying, to convert the extract into a powder, granule, or other suitable form that preserves the stability of the active constituents. The capacity of green tea extract 18 to improve longevity is attributed to multiple mechanisms, including potent free-radical scavenging via antioxidant activity, modulation of cell signaling pathways that govern inflammation and cellular senescence, and potential enhancement of metabolic homeostasis. By mitigating oxidative stress and optimizing metabolic processes, green tea extract 18 has been implicated in promoting cellular integrity and resilience, thereby supporting extended health span when used as part of an overall health regimen. In one embodiment, the green tea extract 18 may be 98% polyphenols, of which 60% is EGCG, and <1% caffeine by weight, a purity level that may be achieved via HPLC.

Ginger root extract 20 may be included in the mixture of natural products 14. Ginger root extract is typically a concentrated preparation derived from the rhizome of the Zingiber officinale plant. The raw material typically is cleaned, dried, and milled prior to undergoing solvent-based extraction using polar or non-polar media, such as ethanol, methanol, water, or supercritical carbon dioxide. The extracted constituents include bioactive compounds such as gingerols, shogaols, and related analogs, which can be standardized to a specific potency. The resultant extract can be further refined via filtration or evaporation techniques to remove residual solvents and unwanted particulates to yield a stable, high-strength formulation 12 that is suitable for diverse delivery modalities. Research indicates that ginger root extract 20 exhibits antioxidant and anti-inflammatory attributes. Such pharmacological actions have been hypothesized to mitigate cellular stress, promote healthy inflammatory responses, and support metabolic homeostasis. Ginger root extract 20 has been shown to inhibit NF-κB signaling and COX-2 expression, which are critical pathways in reducing pro-inflammatory responses and chronic inflammation. By targeting these specific signaling cascades, ginger root extract 20 serves as an anti-inflammatory agent that contributes to longevity by mitigating cellular stress and supporting overall physiological resilience. These properties collectively are thought to play a role in longevity by reducing age-related oxidative damage, modulating molecular signaling pathways, and enhancing overall physiological resilience. In one embodiment, the ginger root extract 20 may be 20% gingerols by weight, a purity level that may be achieved via HPLC.

The mixture of natural products 14 may include rosemary extract 22, which is a concentrated preparation of bioactive compounds derived from the aerial parts of the Rosmarinus officinalis plant, also known as salvia rosmarinus. This plant contains significant quantities of antioxidants such as rosmarinic acid, carnosic acid, and carnosol, which are isolated using solvent-based extraction, supercritical fluid extraction, or steam distillation of dried and milled rosemary leaves, followed by filtration, concentration, and drying processes designed to retain and enrich the phytochemical profile. The principal mechanism by which rosemary extract 22 may contribute to longevity involves its capacity to mitigate oxidative stress and inflammatory pathways, as the phenolic constituents demonstrate robust free-radical scavenging activities, which in turn help maintain cellular integrity, reduce tissue damage, and support homeostatic processes that are critical for healthy aging. In an embodiment, the rosemary extract 22 may be 13% carnosic acid and/or 6% carnosol by weight, a purity level that may be achieved via HPLC.

The mixture of natural products 14 may include resveratrol 24, which is a polyphenolic compound also known by the chemical name trans-3,5,4′-trihydroxystilbene. Resveratrol 24 commonly is found in grapes, berries, peanuts, and the roots of Polygonum cuspidatum and can be produced through direct extraction from these botanical sources. Resveratrol 24 also can be produced by chemical synthesis and by microbial fermentation utilizing engineered yeast or bacterial strains for large-scale manufacture. Resveratrol's 24 longevity-related effects have been associated with its ability to modulate several interlinked molecular pathways, particularly those governing energy sensing, gene transcription, and oxidative stress responses. One prominent mechanism involves the activation of SIRT1, an NAD-dependent deacetylase implicated in the regulation of metabolic processes and stress responses. This may lead to the deacetylation of various transcription factors and coactivators that oversee cellular homeostasis and promote survival under conditions of caloric restriction. Resveratrol 24 also has been documented to influence the activity of AMP-activated protein kinase (AMPK), thereby altering downstream targets involved in glucose and lipid metabolism and potentially modulating mTOR signaling. Additionally, resveratrol 24 inhibits NF-κB, a transcription factor central to inflammatory responses, thereby reducing the expression of pro-inflammatory cytokines and mitigating chronic inflammation associated with aging. This inhibition complements its antioxidant properties, which reduce the reactive oxygen species burden, further enhancing its anti-inflammatory and longevity-promoting effects. In one embodiment, the resveratrol 24 may be 99% trans-3,5,4′-trihydroxystilbene, with the remainder comprising mostly cis-3,5,4′-trihydroxystilbene.

The mixture of natural products 14 may include ursolic acid 26, which is a pentacyclic triterpenoid known by the chemical name 3β-hydroxyurs-12-en-28-oic acid. Ursolic acid 26 may be predominantly present in the epidermal waxes of various fruits, most notably apple peels, as well as in numerous botanical species such as rosemary and holy basil. It generally is produced by solvent-based extraction of these plant materials using alcohols, followed by subsequent fractionation and purification steps, which may include column chromatography or crystallization to achieve the desired chemical purity. Ursolic acid 26 may have beneficial pharmacological effects by modulating pathways linked to aging, including those associated with inflammation, oxidative stress, and metabolic regulation, thereby potentially improving cellular resilience and maintaining physiological homeostasis, which in some models has correlated with extended health span. Additionally, ursolic acid 26 has been shown to influence the PI3K/AKT and MAPK pathways, which are critical for cellular survival, metabolic regulation, and muscle health. These pathways contribute to enhanced mitochondrial function and energy efficiency, further supporting the role of ursolic acid 26 in promoting overall health and longevity. In one embodiment, the ursolic acid 26 may be 90% by weight, a purity level that may be achieved via HPLC.

The mixture of natural products 14 may include isoquercetin 28, which is a naturally occurring flavonoid glycoside derived from quercetin known by the chemical name 3-(β-D-Glucopyranosyloxy)-3′,4′,5,7-tetrahydroxyflavone. Isoquercetin 28 commonly is found in various plant sources including medicinal herbs, fruits, and vegetables is be produced via extraction from these botanical materials followed by purification steps incorporating solvent partitioning, crystallization, or chromatographic separation. Alternatively, isoquercetin 28 may be synthesized by enzymatic glycosylation techniques that selectively attach glucose to the parent quercetin under controlled reaction conditions. Isoquercetin 28 may foster longevity through its potent antioxidant properties that mitigate oxidative stress at the cellular level, as well as through potential anti-inflammatory and cell-signaling modulation effects that can support the maintenance of homeostatic physiological processes over time. In one embodiment, the isoquercetin 28 may be 95% by weight, a purity level that may be achieved via HPLC.

The mixture of natural products 14 may further include luteolin 30, which is a naturally occurring flavonoid classified under the broader category of flavones and is most abundantly found in various plant species, including celery, parsley, thyme, and certain medicinal herbs. Luteolin 30 may be obtained either through extraction from these botanical sources, wherein plant materials are harvested, dried, and subjected to solvent-based or other fractionation techniques to isolate the active compound. Luteolin 30 also can be synthesized from precursor molecules in a multi-step chemical reaction process that yields luteolin 30 at a high purity level. Luteolin 30 may have antioxidant and anti-inflammatory properties, as luteolin 30 is believed to mitigate cellular oxidative stress and modulate key signaling pathways involved in immune regulation, stress resistance, and metabolic homeostasis, thereby promoting healthier aging and potentially prolonging lifespan. Additionally, luteolin 30 has been shown to inhibit the production of pro-inflammatory cytokines such as TNF-α, IL-1β, and IL-6. By directly targeting these cytokines, luteolin 30 plays a pivotal role in reducing chronic inflammation, a key factor in age-related decline and various inflammatory conditions. In one embodiment, the luteolin 30 may be 98% by weight, a purity level that may be achieved via HPLC.

The mixtures of natural products 14 disclosed herein achieve enhanced efficacy through synergistic interactions between the natural products comprising the mixtures of natural products 14, which collectively target oxidative stress, inflammation, metabolic dysregulation, and cellular senescence. For example, resveratrol 24 activates SIRT1 to improve mitochondrial biogenesis and stress resistance, while green tea extract 18 provides antioxidant support that complements resveratrol's 24 effects on cellular signaling pathways such as AMPK and mTOR. Gotu kola extract 16 enhances microcirculatory parameters, amplifying the bioavailability and delivery of other natural products in the formulation 12, while rosemary extract 22 mitigates oxidative damage in a manner that enhances cellular renewal processes. This synergy provides a multifaceted approach to promoting longevity and cellular health.

The mixtures of natural products 14 disclosed herein are believed to synergistically influence key pathways involved in cellular health and longevity, particularly through the modulation of FOXO transcription factors and PGC-1a. Resveratrol 24 activates SIRT1, which subsequently deacetylates and enhances the activity of FOXO transcription factors. This activation promotes the expression of genes responsible for stress resistance, cellular repair, and longevity. Resveratrol 24 further interacts with PGC-la to enhance mitochondrial biogenesis and metabolic efficiency, thereby supporting energy homeostasis and cellular resilience. Additionally, green tea extract 18, which is rich in epigallocatechin gallate (EGCG), is implicated in the modulation of FOXO and PGC-1α pathways, which contributes to its anti-aging and antioxidative effects.

Other components of the mixtures of natural products 14 further enhance these pathways. Ursolic acid 26 has been shown to activate PGC-la, improving mitochondrial function and muscle health, while gotu kola extract 16 indirectly supports FOXO activity by reducing oxidative stress and enhancing cellular repair mechanisms. Isoquercetin 28 and luteolin 30 provide complementary antioxidant and anti-inflammatory effects, further modulating FOXO and PGC-1α-related signaling cascades. Collectively, the mixtures of natural products 14 target these critical regulatory nodes, delivering comprehensive benefits in mitigating age-associated decline, enhancing cellular health, and promoting longevity.

Table 1 below provides ten exemplary mixtures of natural products 14 (Mixtures 1-10) illustrating various dosage ranges and combinations of the natural products disclosed herein. The mixtures of natural products may be blended with the fillers, the flavoring agent, and the preservative in accordance with the procedures described above. It should be understood that the specific weight percentages of Table 1 are non-limiting examples, and alternative ratios or dosages may be selected based on subject population, route of administration, target indication, or formulating preferences. Typical dosage ranges for the mixtures of natural products 14 within the formulation 12 are from 200 mg to 5000 mg and even more typically 250 to 1000 mg.

TABLE 1
Exemplary Examples of the Mixture of natural products 14

| | % Weight of Natural Product in Total Weight of Mixture of Natural Products | | | | | | | |
|---|---|---|---|---|---|---|---|---|
| Mixture | Gotu Kola | Green Tea | Ginger Root | Rosemary | Resveratrol | Ursolic Acid | Isoquercetin | Luteolin |
| 1 | 21.98 | 16.48 | 13.19 | 13.19 | 10.99 | 10.99 | 10.99 | 2.20 |
| 2 | 24.00 | 20.00 | 10.00 | 16.00 | 15.00 | 6.00 | 8.00 | 1.00 |
| 3 | 12.90 | 17.20 | 25.81 | 8.60 | 16.13 | 10.75 | 6.45 | 2.15 |
| 4 | 26.32 | 10.53 | 8.77 | 12.28 | 17.54 | 17.54 | 2.63 | 4.39 |
| 5 | 19.51 | 12.20 | 24.39 | 12.20 | 4.88 | 18.29 | 6.10 | 2.44 |
| 6 | 32.26 | 24.19 | 16.13 | 8.06 | 4.84 | 8.06 | 4.84 | 1.61 |
| 7 | 9.71 | 9.71 | 9.71 | 19.42 | 9.71 | 24.27 | 15.53 | 1.94 |
| 8 | 17.14 | 17.14 | 17.14 | 17.14 | 17.14 | 4.76 | 4.76 | 4.76 |
| 9 | 19.92 | 19.92 | 15.94 | 15.94 | 15.94 | 9.96 | 1.99 | 0.40 |
| 10 | 17.41 | 14.93 | 17.41 | 12.44 | 17.41 | 18.66 | 1.24 | 0.50 |

Each of the exemplary mixtures of natural products 14 described in Table 1 are intended to exhibit synergistic effects among the individual constituents. For instance, the antioxidant activities of green tea extract 18 and ginger root extract 20 can complement the sirtuin-activating properties of resveratrol 24, while the microcirculatory support offered by gotu kola extract 16 potentially enhances the distribution and uptake of ursolic acid 26, isoquercetin 28, and luteolin 30. The mixtures of natural products 14 reflect an understanding that the amounts of each natural product may be modified based on subject population, treatment duration, or specific health goals such as metabolic regulation, cognitive support, or mitochondrial function enhancement. Embodiments incorporating these alternate formulations 12 aim to provide a more comprehensive and adaptable approach to healthy aging, cellular renewal, and long-term systemic well-being.

The mixtures of natural products 14 disclosed in Table 1 are provided for illustration and do not limit the scope of the claims. Different proportions of the natural products or the addition of complementary agents (e.g., vitamins, minerals, or other phytochemicals) may be utilized to create alternative formulations 12 consistent with the present disclosure's objective of modulating key longevity pathways. These examples of the mixtures of natural products 14, in combination with standard pharmaceutical excipients 34 and manufacturing methods, provide broad latitude for tailoring precise nutritional or therapeutic interventions to support cellular health and longevity.

Table 2 provides putative functional activities and highlights the additive and synergistic benefits of each natural product of the mixture of natural products 14. The rows in Table 2 correspond to individual natural products. The columns of Table 2 outline key functional benefits, such as antioxidant activity, anti-inflammatory effects, cellular repair, longevity pathway activation, and cognitive support. Each cell in the table represents the specific contribution of the natural product to a particular functional benefit, which may be qualitative (e.g., “modulates inflammatory pathways”) or quantitative (e.g., “reduces TNF-α by 20%”). Table 2 demonstrates the multifunctional and complementary properties of the formulation 12. For example, resveratrol 24 is shown to activate the SIRT1 pathway, enhance mitochondrial biogenesis, and reduce inflammatory markers such as C-reactive protein (CRP), while green tea extract 18, rich in EGCG, scavenges reactive oxygen species (ROS) and inhibits mTOR signaling. Similarly, gotu kola extract 16 enhances microcirculatory parameters and collagen synthesis, which support tissue repair and cellular vitality.

TABLE 2
Functional Activities of the Natural Products of the Mixture of Natural Products 14

| | | | | Longevity | |
|---|---|---|---|---|---|
| Natural | Antioxidant | Anti- | Cellular | Pathway | |
| product | Activity | Inflammatory | Repair | Activation | Cognitive Support |
| Gotu kola | Increases SOD | Reduces TNF- | Enhances | Indirectly | Improves memory |
| extract 16 | activity by | α by 20% | collagen | supports | retention |
| | 30% (Ref. X) | (Ref. X) | synthesis | FOXO | (Ref. X) |
| | | | | activation | |
| | | | | (Ref. X) | |
| Green tea | High EGCG | Reduces IL-6 | N/A | Activates | Enhances |
| extract 18 | scavenges ROS | by 25% | | AMPK, | neuroplasticity |
| | (Ref. Y) | (Ref. Y) | | inhibits | (Ref. Y) |
| | | | | mTOR | |
| | | | | (Ref. Y) | |
| Ginger root | Modulates | Reduces NF- | Promotes | Increases | N/A |
| extract 20 | NOX enzymes | κB signaling | tissue healing | mitochondrial | |
| | (Ref. Z) | (Ref. Z) | | biogenesis | |
| | | | | (Ref. Z) | |
| Rosemary | High | Reduces COX- | Mitigates | Supports | N/A |
| extract 22 | rosmarinic | 2 expression | DNA damage | PGC-1α | |
| | acid content | (Ref. Z) | | pathway | |
| | | | | (Ref. Z) | |
| Resveratrol | Activates | Lowers CRP | Enhances | Activates | Protects against |
| 24 | NRF2 | levels by 15% | mitochondrial | SIRT1, boosts | neuronal stress |
| | (Ref. W) | (Ref. W) | repair | NAD+ | (Ref. W) |
| | | | | (Ref. W) | |
| Ursolic acid | Reduces ROS | Suppresses | Reduces | Activates | N/A |
| 26 | generation | pro- | muscle | PGC-1α | |
| | | inflammatory | atrophy | pathway | |
| | | cytokines | | (Ref. W) | |
| | | (Ref. W) | | | |
| Isoquercetin | Neutralizes | Modulates | Supports | N/A | N/A |
| 28 | peroxynitrite | cytokine | endothelial | | |
| | radicals | release | repair | | |
| | | (Ref. X) | | | |
| Luteolin 30 | Inhibits | Blocks NF-κB | Mitigates | Enhances | Improves synaptic |
| | oxidative | pathways | cellular | FOXO activity | plasticity |
| | damage | (Ref. X) | senescence | (Ref. X) | (Ref. X) |

Table 2 illustrates that each natural product contributes to multiple overlapping pathways that are critical for mitigating the effects of aging and supporting systemic health. For example, the combined antioxidant effects of green tea extract 18 and luteolin 30, coupled with the anti-inflammatory properties of resveratrol 24 and ginger root extract 20, result in enhanced protection against oxidative stress and chronic inflammation. This synergistic activity is further complemented by the cellular repair mechanisms supported by gotu kola extract 16 and isoquercetin 28, which improve tissue integrity and endothelial function.

The longevity pathway activation column highlights the ability of specific natural products, such as resveratrol 24, green tea extract 18, and ursolic acid 26, to influence critical molecular pathways including SIRT1, AMPK, and PGC-la. These pathways play essential roles in enhancing mitochondrial function, reducing senescence, and improving metabolic efficiency. Additionally, cognitive support is achieved through the neuroprotective and neuroplasticity-enhancing effects of the natural products such as gotu kola extract 16, green tea extract 18, and luteolin 30, which are particularly relevant for mitigating age-related cognitive decline.

The mixtures of natural products 14, as disclosed in Table 1, exhibit extensive synergistic effects by modulating critical biochemical pathways that collectively address the hallmarks of aging and enhance systemic health. Each natural product contributes uniquely to reducing oxidative stress, mitigating chronic inflammation, and improving cellular and metabolic functions, while their combined effects amplify these benefits through interlinked molecular mechanisms.

The mixtures of natural products 14 are uniquely positioned to ameliorate numerous hallmarks of aging, leveraging the synergistic effects of its components to address oxidative stress, inflammation, mitochondrial dysfunction, genomic instability, telomere attrition, and cellular senescence. For example, green tea extract 18 and rosemary extract 22 activate the Nrf2 pathway, bolstering the antioxidant defense system to mitigate oxidative damage and enhance genomic stability. Gotu kola extract 16 promotes telomerase activity and collagen synthesis, improving cellular repair and tissue resilience. Resveratrol 24 and ursolic acid 26 inhibit mTOR signaling and enhance AMPK activation, collectively reducing cellular senescence and improving energy metabolism. Ginger root extract 20 and luteolin 30 reduce chronic inflammation by inhibiting NF-κB signaling and suppressing pro-inflammatory cytokines such as TNF-α, IL-6, and IL-1β. Isoquercetin 28 contributes to endothelial repair and vascular function, while also enhancing detoxification through the upregulation of phase II detoxifying enzymes. Collectively, the composition comprehensively supports systemic health, mitigating the cumulative effects of aging by addressing multiple interconnected biochemical and physiological pathways.

For example, Nrf2 activation, facilitated by multiple components such as green tea extract 18, rosemary extract 22, and ginger root extract 20, promotes the upregulation of antioxidant enzymes, including superoxide dismutase (SOD), catalase, and glutathione peroxidase, thereby reducing oxidative damage and enhancing cellular resilience. Simultaneously, inhibition of the NF-κB pathway by resveratrol 24, luteolin 30, and ginger root extract 20 attenuates the expression of pro-inflammatory cytokines such as TNF-α, IL-6, and IL-1β, reducing chronic inflammation and its associated physiological decline.

The mixtures of natural products 14 further targets energy metabolism and cellular homeostasis through activation of the AMPK pathway by the natural products like green tea extract 18 and ursolic acid 26, which regulate glucose and lipid metabolism, enhance mitochondrial biogenesis, and improve insulin sensitivity. Concurrently, inhibition of the mTOR pathway by green tea extract 18 and ursolic acid 26 supports cellular autophagy and reduces senescence, contributing to anti-aging effects.

Additionally, neuroprotective effects are achieved through mechanisms such as enhanced cerebral blood flow promoted by gotu kola extract 16, increased levels of neurotrophic factors like brain-derived neurotrophic factor (BDNF) from rosemary extract 22, and reduced neuroinflammation mediated by luteolin 30 and resveratrol 24. These effects collectively support cognitive function, mitigate neurodegenerative risks, and promote mental resilience.

The mixtures of natural products 14 also exhibits chemo-preventative and detoxifying properties by modulating apoptosis pathways and enhancing the activity of phase II detoxifying enzymes. Isoquercetin 28, for instance, promotes the elimination of toxic substances while inducing apoptosis in aberrant cells. This is complemented by the lipid-lowering effects of ursolic acid 26 and rosemary extract 22, which improve cardiovascular health by regulating cholesterol and promoting nitric oxide-mediated vasodilation.

Synergy among the constituent natural products is further evident in their ability to modulate gut microbiota composition, enhancing gastrointestinal health and systemic immune function. For example, ginger root 108 and isoquercetin 28 support the growth of beneficial bacteria, contributing to improved nutrient absorption, metabolic regulation, and inflammation control.

In use, the multi-ingredient natural products composition 10 enables a method 38 of treating a subject with a mixture of natural products 14 to improve a biological process in the subject. The method 38 entails provision of a mixture of natural products 14 comprising at least five natural products selected from the group consisting of gotu kola extract 16, green tea extract 18, ginger root extract 20, rosemary extract 22, resveratrol 24, ursolic acid 26, isoquercetin 28, and luteolin 30. The method also entails provision of one or more of a liquid 32, an excipient 34, and a device 36 configured to render the mixture of natural products 14 suitable for therapeutic delivery to a subject. Upon producing a formulation 12, it can be delivered to the subject to improve the biological process.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Therefore, the foregoing is considered as illustrative only of the principles of the disclosure. Further, since numerous modifications and changes will readily occur to those skilled in the art, it is not desired to limit the disclosure to the exact combinations, methods, and operations shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A multi-ingredient natural products composition comprising:
a formulation comprising:
a mixture of natural products comprising at least five natural products selected from the group consisting of gotu kola extract, green tea extract, ginger root extract, rosemary extract, resveratrol, ursolic acid, isoquercetin, and luteolin; and
one or more of a liquid, an excipient, and a device configured for rendering the mixture of natural products suitable for therapeutic delivery to a subject; and
wherein the formulation is configured for improving a biological process in the subject.

2. The multi-ingredient natural products composition of claim 1, wherein, if present in the formulation, each natural product comprises by weight of a total percentage of the mixture of natural products:
5.0% to 50.0% gotu kola extract;
5.0% to 30.0% green tea extract;
5.0% to 30.0% ginger root extract;
5.0% to 25.0% rosemary extract;
2.0% to 25.0% resveratrol;
2.0% to 30.0% ursolic acid;
0.5% to 20.0% isoquercetin; and
0.2 to 6.0% luteolin.

3. The multi-ingredient natural products composition of claim 1, wherein, if present in the formulation, each natural product comprises by weight of a total percentage of the mixture of natural products:
9.0% to 33.0% gotu kola extract;
7.0% to 26.0% green tea extract;
7.0% to 27.0% ginger root extract;
7.0% to 20.0% rosemary extract;
4.0% to 20.0% resveratrol;
4.0% to 25.0% ursolic acid;
1.0% to 16.0% isoquercetin; and
0.4 to 5.0% luteolin.

4. The multi-ingredient natural products composition of claim 1, wherein dose the mixtures of natural products delivered from the formulation is from 200 mg to 5000 mg.

5. The multi-ingredient natural products composition of claim 1, wherein dose the mixtures of natural products delivered from the formulation is from 250 mg to 1000 mg.

6. The multi-ingredient natural products composition of claim 1, wherein the one or more of a liquid, an excipient, and a device renders the formulation deliverable to the subject as a solution, emulsion, or a suspension by oral delivery, parenteral delivery, topical delivery, or inhalational delivery.

7. The multi-ingredient natural products composition of claim 1, wherein the one or more of a liquid, an excipient, and a device renders the formulation deliverable to the subject orally as a pill, a capsule, a time-release capsule, or a tablet.

8. The multi-ingredient natural products composition of claim 1, wherein the one or more of a liquid, an excipient, and a device renders the formulation deliverable transdermally to the subject.

9. The multi-ingredient natural products composition of claim 1, wherein, if present in the mixture of natural products, each natural product includes:
the gotu kola extract includes at least 35% asiaticosides by weight;
the green tea extract includes at least 95% total polyphenols by weight with 55% of the polyphenols being epigallocatechin gallate and <1% caffeine by weight;
the ginger root extract includes at least 15% gingerols by weight;
the rosemary extract includes at least 10% carnosic acid and/or 4% carnosol by weight;
the ursolic acid has a weight purity of at least 85%;
the isoquercetin has a weight purity of at least 90%; and
the luteolin has a weight purity of at least 94%.

10. A method of treating a subject with a mixture of natural products to improve a biological process in the subject, the method comprising:
providing of the mixture of natural products comprising at least five natural products selected from the group consisting of gotu kola extract, green tea extract, ginger root extract, rosemary extract, resveratrol, ursolic acid, isoquercetin, and luteolin;
providing one or more of a liquid, an excipient, and a device configured to render the mixture of natural products suitable for therapeutic delivery to a subject;
combining the mixture of natural products and the one or more of the liquid, the excipient, and the device to generate a formulation; and
delivering the formulation to the subject to improve the biological process.
